(19) United States
(12) Patent Application Publication
(10) Pub. No.: US 2006/0224050 A1
(43) Pub. Date: Oct. 5, 2006

(54) Refrigerator for health care

(76) Inventors: Ik Kyu Lee, Gimhae-si (KR); Seung Jin Oh, Daegu (KR)

Correspondence Address: FLESHNER & KIM, LLP, P.O. BOX 221200, CHANTILLY, VA, 20153, US

(21) Appl. No.: 11/391,697

(22) Filed: Mar. 29, 2006

(30) Foreign Application Priority Data
Mar. 30, 2005 (KR) P2005-0026464
May 23, 2005 (KR) P2005-0042968
May 23, 2005 (KR) P2005-0042970
May 23, 2005 (KR) P2005-0042972

(51) Int. Cl. A61B 5/00; A61B 5/02

(52) U.S. Cl. 600/300; 600/921; 128/923; 128/920

(57) ABSTRACT

A refrigerator for health care is disclosed. A refrigerator for health care comprising a display device for displaying user health data; and a controller for receiving user health data from outside and supplying the user health data to the display device. The refrigerator according to the present invention has an effect that the user can take care of his/her health more efficiently.

This application claims the benefit of the Patent Korean Application No.P2005-0026464, filed on Mar. 30, 2005, No.P2005-0042968 filed on May 23, 2005, No.P2005-0042970 filed on May 23, 2005 and No.2005-0042972 filed on May 23, 2005, which is hereby incorporated by reference as if fully set forth herein.

### BACKGROUND OF THE INVENTION

1. Field of the Invention

The present invention relates to a refrigerator, and more particularly, to a refrigerator for health care which can take care of a user's health efficiently.

2. Discussion of the Related Art

Generally, a refrigerator is a device that keeps food fresh in a low temperature state for a long term by maintaining a freezer chamber and a cold storage chamber at low temperatures using chilly drafts, respectively. Thus, refrigerators are globally provided according to massive demand and supply due to the development of the industrial society.

Meanwhile, most of diseases in the modern industrial society mostly result from wrong eating habits, as well as partially from such an indirect influence as environmental pollution. Especially, adult diseases due to obesity have been increasing nowadays.

Since those adult diseases mostly rise from bad eating habits, it is necessary in the modern society to regularly check people's health data such as blood sugar or blood pressure, let alone to improve eating habits.

According to ‘Sasang Medicine’, in other words, a 4-constitutional-medicine which is developed and originated in oriental medicine, everybody eats the same food but there are differences. Thus, individual constitutional type is analyzed to perceive diseases which each individual is liable to. Hence, food and eating habits should be managed to prevent those diseases.

Consequently, there have been demands for refrigerators having an all-around health care function for a user.

### SUMMARY OF THE INVENTION

Accordingly, the present invention is directed to a refrigerator for health care that substantially obviates one or more problems due to limitations and disadvantages of the related art.

An object of the present invention is to provide a refrigerator for health care which can take care of a user's health.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve these objects and other advantages and in accordance with the purpose of the invention, as embodied and broadly described herein, a refrigerator for health care comprising: a display device for displaying user health data; and a controller for receiving user health data from outside and supplying the user health data to the display device.

The health data includes data relating to at least one of a user's weight, body type, face image, body constitution and blood pressure. Preferably, the controller comprises a data processing part for changing the health data into health information useful for health care.

The health information changed by the data processing part includes information relating to at least one of a user health condition gained by comparison between the health data and predetermined standard data, a diet corresponding with the health data, and an exercising method corresponding with the health data.

The refrigerator for health care according to the present invention further includes a measure device for measuring user health data and supplying measured health data to the controller. The measure device comprises a photographing part for receiving a user face image.

The measure device includes a blood sugar sensor for measuring a user blood sugar level. Preferably the blood sugar sensor is a not-invasive blood sugar sensor. Also, preferably, the blood sugar sensor is provided on the refrigerator door.

On the other hand, the refrigerator for health care according to the present invention may further include a terminal for connecting an external measure device thereto.

The controller determines whether the external measure device is connected to the terminal, and receives the health data form the external measure device, once the external measure device is connected to the terminal.

Here, preferably, the terminal includes a USB port (Universal Serial Bus).

The refrigerator for health care according to the present invention may further include a diagnosis information transmitting/receiving part for transmitting the health data to the diagnosis server through the network and for receiving the health diagnosis information performing/filling a diagnosis/prescription for the user through the diagnosis server based on the health data.

Here, the health information displayed by the display device includes health diagnosis information received from the diagnosis information transmitting/receiving part.

The refrigerator for health care according to the present invention may further include a data inputting part for receiving body data inputted by the user. The controller includes a data storing part for accumulatively storing the health data. The health data stored in the data storing part is classified and stored based on respective user information.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a front sectional view illustrating a refrigerator according to a first embodiment of the present invention.

FIG. 2 is a block view of FIG. 1.

FIG. 3 is a flow chart illustrating a method for controlling the refrigerator according to FIG. 1.

FIG. 4 is a front sectional view illustrating a refrigerator according to a second embodiment of the present invention.

FIG. 5 is a block view of FIG. 2.

FIG. 6 is a flow chart illustrating a method for controlling the refrigerator according to FIG. 2.

FIG. 7 is a front sectional view illustrating a refrigerator according to a third embodiment of the present invention.

FIG. 8 is a block view of FIG. 7.

FIG. 9 is a flow chart illustrating a method for controlling the refrigerator according to FIG. 7.

FIG. 10 is a diagram schematically illustrating a remote health check-up system of a refrigerator according to a fourth embodiment of the present invention.

FIG. 11 is a block diagram of FIG. 10.

FIG. 12 is a flow chart illustrating a method for controlling the refrigerator of FIG. 10.

### DETAILED DESCRIPTION OF THE INVENTION

Reference will now be made in detail to the preferred embodiments of the present invention, examples of which are illustrated in the accompanying drawings. Wherever possible, the same reference numbers will be used throughout the drawings to refer to the same or like parts.

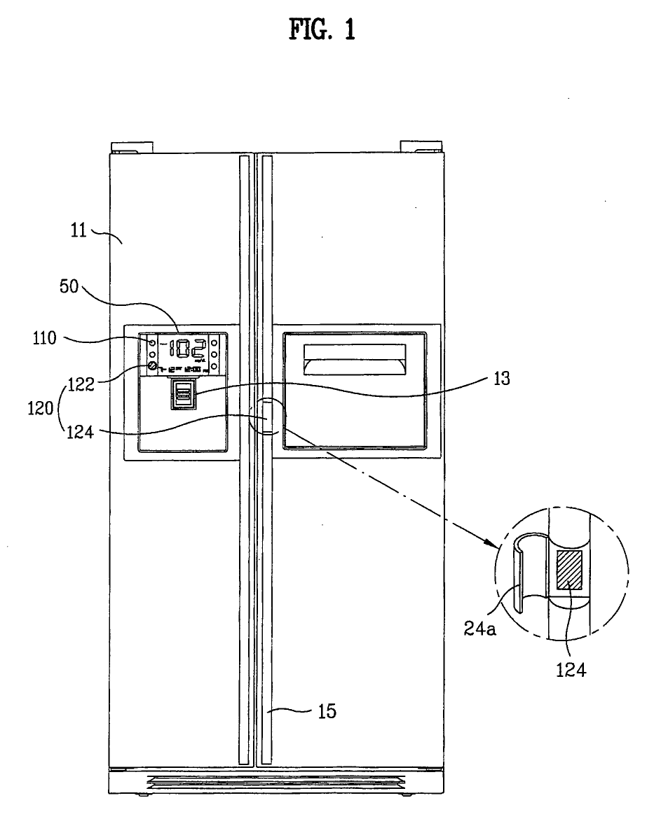

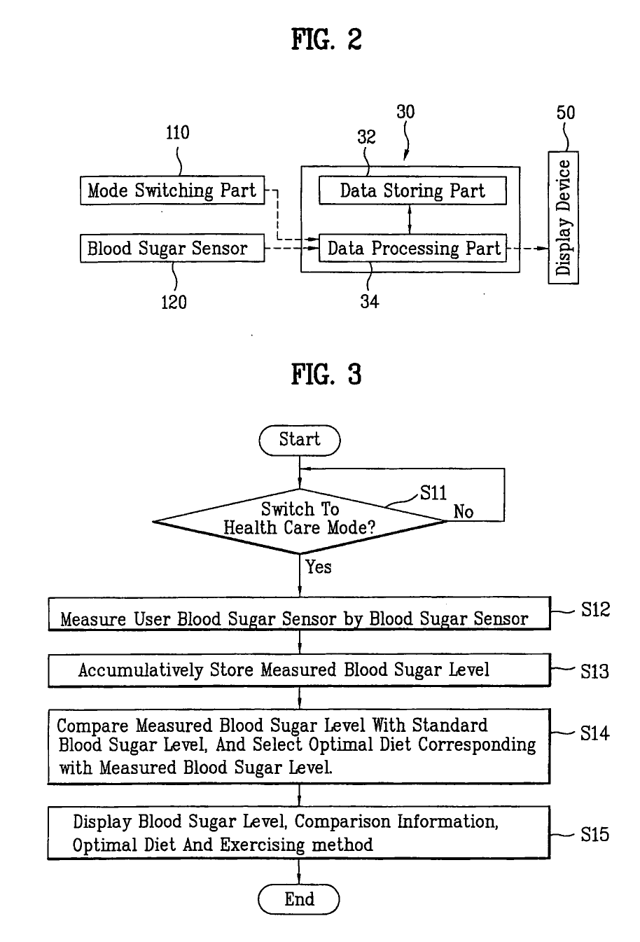

Referring to FIGS. 1 and 2, a refrigerator according to a first embodiment of the present invention will be described.

In general, a refrigerator includes a refrigerator body 100 having a freezing chamber, a refrigerating chamber, a pair of doors 11 and a controller 30. The pair of doors 11 is rotatably coupled to both sides of the refrigerator door 100, and the controller 30 is provided within the refrigerator body. An input button for adjusting the operation of refrigerator and a display part 50 for displaying the operation state of refrigerator are further included on the refrigerator door 11.

A dispenser 13 is provided on the refrigerator door 11 for supplying ice or cold water on a user's sake.

The refrigerator according to a first embodiment of the present invention includes a measure device measuring a user's health data, a display device 50 displaying the health data measured in the measure device and a controller 30 to health care receiving the user's health data and supplying it to the display device 50.

The health data may be various kinds of data relating to a user's health such as a user's weight, constitution type, face shape, body composition, and blood pressure. The measure device measures the user health data.

According to the first embodiment, the measure device is a blood pressure sensor 120 to measure a user blood pressure data amount the various kinds of data.

The pressure sensor 120 is a sensor measuring a user blood pressure, and preferably a non-invasive blood sugar sensor which has been developed recently. The sensor is capable of determining blood sugar level non-invasively. More specifically, according to the non-invasive blood sugar sensor measures the user blood pressure by using that micro waves are injected to blood currents such that the returning electromagnetic waves are different based on blood sugar level.

Generally, if a blood sugar level in an empty stomach is more than 140 mg of 1 dl, or more than 200 mg of 1 dl after drinking water containing 75 g in spite of less than the value of 140 mg of 1 dl, it is diagnosed as diabetes. Thus, a diabetic had better take care of his/her blood sugar level by a diet or medicine to lower less than 120 mg of 1 dl in an empty stomach without breakfast, or less than 150 mg of 1 dl after a meal.

Though the blood sugar sensor may be provided in several portions, preferably the blood sugar sensor is provided at the handle portion 15 of refrigerator door or on a control panel for facilitating the access thereto.

It is embodied in this embodiment that the number of blood sugar sensor 120 is two. And one of the two is provided at an adjacent portion to input buttons in the control panel and the other of the two is provided on the handle portion 15 of refrigerator door. Preferably, the blood sugar sensor 120 is provided high enough to allow a user each access thereto.

Meanwhile, a sensor cover 124a provided in the blood sugar sensor opens/closes the blood sugar sensor 124, such that the blood sugar sensor 124 is exposed outside only when the user blood sugar is measured. The sensor cover 124a is opened/closed, because it has a first side thereof hingedly connected with the handle portion 15 of refrigerator door and a hook formed at a second side thereof. Alternatively, the sensor cover may be slidingly opened and closed upwardly/downwardly.

The display device 50 displays health data measured by the blood sugar sensor 120 and commonly is a LCD 50 (Liquid Crystal Display). Particularly, the display device 50 may display the measured blood sugar in various ways such as evaluated figures, graphs or various types of characters.

The controller 30 receives and supplies the measured health data to the display device 50. The controller 30 not only transmits health data from the blood sugar sensor to the display device 50, but also changes the health data measured in the blood sugar sensor 120 into health information useful to health care and then supplies the health information to the display device 50.

Thus, the controller 30 transmits data relating to the user blood sugar level of blood sugar sensor 120, as well as user health condition based on comparison between the measured blood sugar level and a standard blood sugar level, to the display device 50.

The controller 30 includes a data processing part 34 for transmitting the measured health data of blood sugar sensor 120 to the display device 50, followed by changing the health data into useful health data for health care, and a data storing part 32 for storing at least one of the health data changed by the data processing part 34.

The data storing part 32 stores each blood sugar level measured by the blood sugar sensor 120 accumulatively, such that a gauge of change in each user's blood sugar level may be provided through the accumulatively stored blood sugar levels. The gauge of change in each user's blood sugar level is displayed in graphs.

Herewith, various kinds of health diets or cooking recipes and exercising methods for the health diets are provided stored in the data storing part 32. Thereby, the user can select the optimal diet and exercising method based on the comparison information between the user blood sugar level and the standard blood sugar level compared by the data processing part 34.

Meanwhile, the first embodiment of the present invention further includes a mode switching part 110 is provided on the control panel.

The mode switching part 110 switches a temperature management mode managing the temperature of the freezing chamber and refrigerating chamber into a health care mode displaying health information based on the user health data, and also the mode switching part 110 may switch various modes.

Although a health care mode is selected by the mode witching part 110 and switched into the health care mode, the temperature of each freezing and refrigerating chamber should be controlled consistently based on values predetermined in the temperature management mode.

It is preferred in mode switching part 110 that buttons on the control panel are shared to switch the operation of refrigerator in the temperature management mode, but an additional button may be provided.

The first embodiment presents the mode switching part 110 for switching modes. Alternatively, it is determined whether the blood sugar sensor 120 is touched by the user. Hence, once the blood sugar sensor 120 is touched, a temperature management mode may be automatically switched into a health care mode.

On the other hand, though not illustrated, the refrigerator for health care may include a data input part for receiving various user data such as a user's sex, age and weight. Hence, an appropriate diet may be selected based on a day calorie according to a standard weight from inputted data, as well as the user blood sugar information measured by the blood sugar sensor 120.

Commonly, the day calorie for an overweight adult is 20˜25 kcal, the day calorie for a light worker (an office worker) is 30 kcal, the day calorie for a middle worker is 35˜40 kcal and the day calorie for a heavy worker is 40˜45 kcal. For example, since a standard weight for a light worker in 165 cm and 80 kg is 60 kg, he/she should intake 20˜25 kcal per 1 kg and 1200˜1500 kcal per day to lose some weight.

Preferably, when planning the diet in the data processing part 34, the diet should include vitamins, minerals as well as major three nutrients of sugar, protein and fat so as to balance the diet.

Referring to FIG. 3, a method for controlling a refrigerator according to the first embodiment of the present invention will be described.

First, a user switches to a health care mode through a mode switching part 110, such that his/her health is managed (S11), and then a user blood sugar level is measured by a blood sugar sensor 120 (S12).

Hence, the measured blood sugar level is accumulatively stored (S13), and an optimal diet corresponding with the measured blood sugar level is selected based on comparing the measured blood sugar level with a standard blood sugar level (S14).

The optimal diet includes all the variously different nutrients within the total calories range based on a day calorie as well as the measured blood sugar level information, because the user's sex, age and weight are additionally inputted with the measured blood sugar level. Also, the diet may be selected from the pre-inputted diets and the diet may be re-configured.

Next, the measured blood sugar level, the comparison information gained by comparing the blood sugar level and the standard level and the optimal diet selected corresponding with the measured blood sugar level are displayed by the display device 50 (S15). Here, preferably two and more optimal diets are selected, such that the user may select one of the two.

Also, the comparison information and the change of user blood sugar level may be displayed in graphs, such that the user can take care of and analyze his/her blood sugar level change more easily.

Together with that, since not only the optimal diet but also the recipes thereof are displayed at the display device 50 through letters or pictures, the user can have diets which may prevent and take care of diseases such as diabetes.

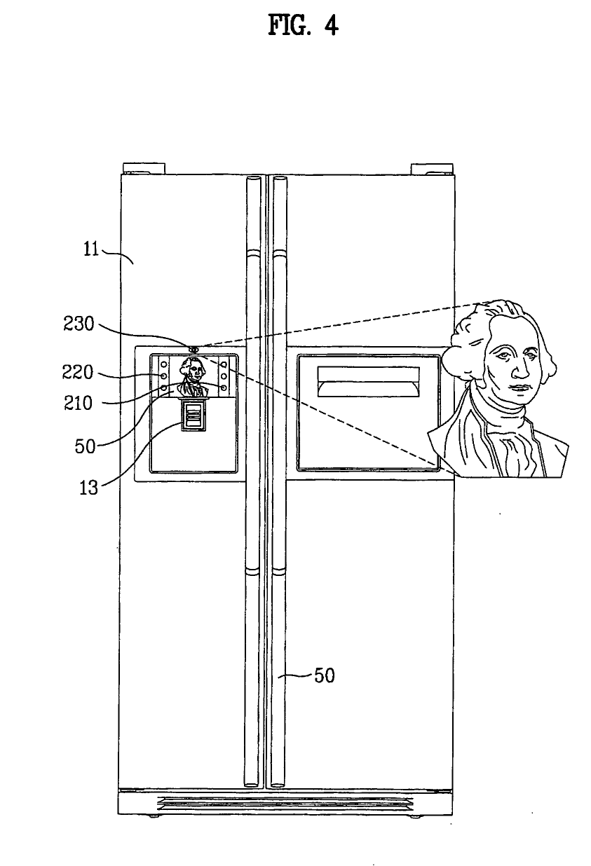

Referring to FIGS. 4 and 5, a refrigerator according to a second embodiment of the present invention will be described.

Like the first embodiment, the second embodiment includes a measure device, a display device 50 and a controller 30.

According to the second embodiment, a photographing part 230 is included for receiving user face images.

The photographing part 230 is a kind of a digital camera for receiving user face images. The photographing part 230 is provided in the upper portion of display device 50 as high to the user's eyes. Preferably, the photographing part 230 is rotatable in an up/down direction and a right/left direction to take an exact face image even when the user stands in various spots.

It is preferred that there may be a virtual frame line to take a face image in a particular range and to judge a body constitution exactly. Also it is preferred that the photographing part 230 may adjust angles and distances for allowing a user face imaged in the virtual frame.

Hence, the controller 30 includes a data processing part 34 and a data storing part 32, and a 4-body constitution information is pre-memorized therein, which may be analyzed according to various face images and various birth information.

The data processing part 34 compares both of the face image of the photographing part 230 and the birth information of the data processing part 220 with the various information memorized in the data storing part 32, such that it is judged which type of the 4-body constitution types the user belongs to.

Alternatively, the user may input his/her body constitution type directly among several body constitution types, instead of judging the user body constitution type through the face image taken by the photographing part 230.

Information on following 4-body constitution types are numbered and stored. Appropriate food and bad food as well as constitutional characteristics of each type corresponding thereto are stored.

The 4-body constitution types, which is said to be judged by face shapes according to Oriental medicine, are classified into “Taeyangin”, “Taeumin”, “Soyangin” and “Soumin”. Face shapes, physical characteristics and appropriate food are following.

First, a “Taeyangin” type person has a big round head and a developed neck and back head, a fast jaw area and small eyes. Also, his/her pulmonary function is good, whereas his/her liver function is not good. In addition, the Taeyangin type person may not walk so long, may have a keen sense of hearing, and may urinate a lot. Furthermore, Taeyangin type women might have tendencies of infertility even though they are healthy.

Good food for a “Taeyangin” type person may be actinidia argutas, clams, crucian carps, cuttlefish, grapes, quinces, apples, buckwheat and nonglutinous rice. The more vegetables they have, the better. They had better avoid fatty food. Whereas, bad food for a Taeyangin type person may be fatty food such as beef and pork, and spicy food such as garlic, radishes, sugar and pepper.

Next, “Taeumin” type person may have a round or oval face, and a big ear, eye, mouth and nose. Also, they may have thick lips. They may have a good liver function, whereas not so good functions in lung, heart, a large intestine and a skin. They may have a keen sense of smelling, and a Taeumin type woman may tends to have chapped hands in winter.

Good food for a “Taeumin” type person may be beef, radish, mushrooms, beans, sweet potatoes, lotus roots, taros, ballflowers, yams, glutinous rice, seaweeds, layer, walnuts, pumpkins, plums, peanuts and pitches. Whereas, bad food for a “Taeumin” type person may be pork, chicken, eggs, goat meat, dog meat, apples and cabbages.

Next, a “Soyangin” type person may have a head protruding both in the frontward and backward or a round head, and have a cheerful look. He/she may have a sharp jaw, not so big mouse and sharp eyes. His/her kidney function is not so good, and he/she may have much heat in body. They are very digestive and not so sweaty. Also, they have a keen sense of seeing. “Soyangin” type men might lacks stamina, and “Soyangin” type women might have tendency of infertility.

Good food for a “Soyangin” type person may be pork, duck, snake-headed fish, terrains, sea sluggers, ear shells, green grams, red beans, millet, barley, melons, tomatoes, strawberries, dropwort, egg plants, cabbages, cucumbers, pumpkins, burdocks and potatoes. Bad food for a “Soyangin” type person may be beef, chicken, peanuts, milk and honey.

A “Soyangin” type person may have a good looking. He/she may have a not-so-big ear, eye, mouth and nose, and also thin lips. His/her face shape looks weak in the eyes. His/her kidney function might good, and most of that type people may not have much sweat. He/she has a keen sense of taste and has a soft skin but not chapped hands. They tend to breathe with a sigh unconsciously.

Good food for a “Soumin” type person may be dog, lamb, goat, chicken, pheasant, hare meat, eel, mandarin fish, anchovy, rice, wild sesame, cabbages, Korean leeks, green onions, carrots, spinach, potatoes and honey. Bad food for a “Soumin” type person may be buckwheat, barley, mung beans, red beans, sweet potatoes, chestnuts, walnuts, pumpkins, watermelon, pears, cucumbers and melons.

Of course, since standard face images are numbered and inputted in the data storing part 32 and also a user face image is photographed and numbered, the data processing part 34 compares the photographed face images with the standard face images to judge which type the user belongs to. Hence, physical characteristics as well as good food are displayed on the display device 50 to notify the user that information.

Here, the data processing part 34 determines which body constitution type the user belong to based on the photographed face images by the photographing part 230, and uses the information such as the user's birth inputted by the data inputting part 220 for exact determination of body constitution type.

The data inputting part 220 is provided with buttons to receive the user's birth, separately with the face images photographed inputted by the photographing part 230.

The display device 50 displays images of the photographing part 230, such that the user can see his/her image and have the image taken. Thereby, exact face image can be taken. Also, the display device 50 displays the determined body constitution information and the appropriate diet for the determined body constitution type.

Especially, the display part 50 is adjusted by the controller 30 to display the determined body constitution and the information thereof by using letters and avatars. Furthermore, even diets and recipes appropriate to the determined body constitution type are displayed by using letters and pictures.

Meanwhile, the refrigerator according to the second embodiment of the present invention includes a mode switching part 210.

Like the first embodiment, the mode switching part 210 switches from a temperature management mode managing the temperature of the freezing as well as the refrigerating chamber to a health care mode displaying a health condition based on user physical information.

Once switched into the health care mode by the mode switching part 210, the photographing part 230 is prepared to photograph a user face image and the display device 50 also is prepared to display the image of photographing part 230.

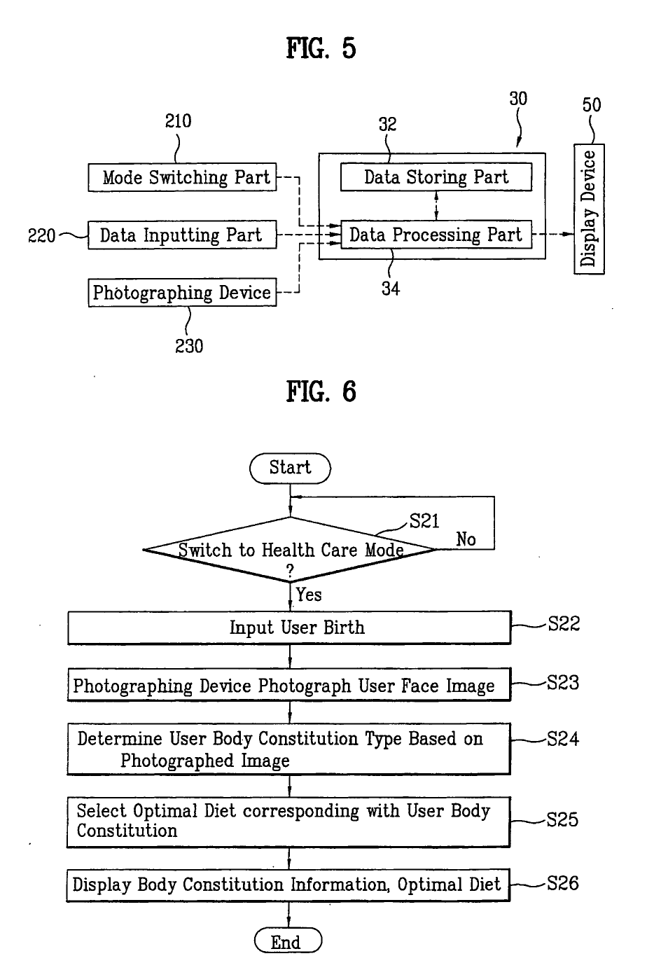

Referring to FIG. 6, a method for controlling the refrigerator according to the second embodiment of the present invention will be described.

First, it is determined whether a temperature management mode is switched to a health care mode (S21). Hence, once the mode being switched to the heath care mode, user birth information is inputted by the data inputting part 220 (S22).

Together with that, a user face image is photographed by the photographing part 230, and then it is determined based on the comparison between the photographed user image and the user birth which body constitution type the user belongs to (24).

Preferably, the photographing part 230 may adjust a photographing angle and photographing distance to take user face images accurately, and also the display device 50 may have a virtual frame lines displayed thereon to include the photographed face image within the virtual frame lines.

Hence, an optimal diet corresponding with the determined user body constitution is selected (S25), and the user body constitution information as well as the optimal diet may be displayed by the display device 50 (S26).

At this time, the display device 50 may display the user body constitution information and the optimal diet in several ways such as letters, pictures and characters.

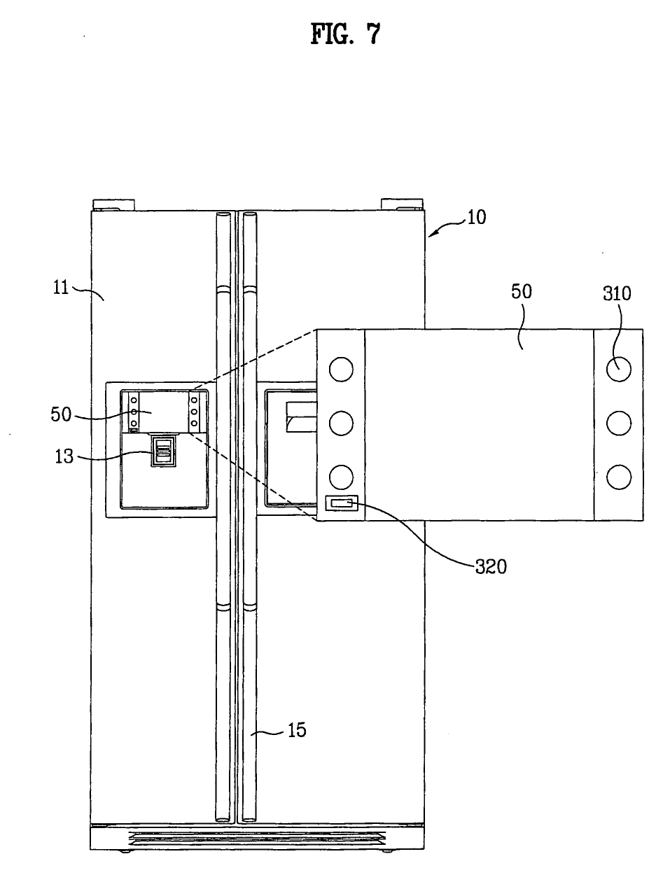

Referring to FIGS. 7 and 8, a refrigerator according to a third embodiment of the present invention will be described.

As described in the above embodiments, the refrigerator according to the third embodiment includes a controller 30 and a display device 50.

However, according to this embodiment, a measure device is not provided on the refrigerator, and an external measure device (A) connected to the refrigerator is provided for gaining user health data.

Thus, the refrigerator according to the third embodiment includes a terminal 320 having the external measure device (A) connected thereto. Thus, the external measure device (A) is connected to the terminal 320 for transmitting measured data to the refrigerator.

That is, according to the third embodiment, the refrigerator 10 and the external measure device (A) are provided, and the external measure device (A) is connected to the terminal 320 of the refrigerator 10, such that the user health data is transmitted to the controller.

The terminal 320 may have the external measure device (A) connected thereto, and the controller 30 determines whether the external measure device (A) is connected to the terminal 320. Hence, once it is determined that the external measure device (A) is connected, the controller receives the health data from the external measure device (A).

The terminal 320 may be provided in several ways, and the this embodiment presents that the terminal 320 is a USB (Universal Serial Bus), which is a kind of an external electric device, for transmitting electric signals between the external measure device (A) and the controller 30.

Preferably, the external measure device (A) is a medical device which may measure blood pressure and BMI to prevent as well as to take care of diseases such as high blood pressure and hyperlipidemia caused by obesity. The external measure device (A) may not only measure blood pressure and BMI, but also may have a USB cable connected to a side thereof.

Of course, it is determined by the controller 30 whether the external measure device (A) is connected to the terminal 320. Hence, once the external measure device (A) is connected, the user body information is transmitted from the external measure device (A).

The controller 30 includes a data processing part 34 and a data storing part 32. The data processing part 34 determines whether the external measure device (A) is connected to the terminal 320, and once the external measure device is connected, the controller receives the body data. Hence, the controller 30 compares the received data with a standard data to judge a user health condition. After that, the judged health condition and the various data are transmitted to the display device 50.

Also, the data storing part 32 stores various standard data, and the data processing part 34 stores comparison information between the user health data and the standard data.

The data storing part 32 not only stores the standard health data and the health condition, but also accumulatively stores and manages various measuring data. Thus the determined health conditions are stored and managed according to each user.

Alternatively, the data storing part 32 may automatically delete the measured body data which have passed a predetermined time period or the body data except the setting numbers

For example, a standard highest blood pressure range is 140˜100 mm Hg and a standard lowest blood pressure range is 90˜70 mm Hg in the data storing part 32. Hence, a health condition is stored in the data managing part based on the standard highest blood pressure range and the standard lowest blood pressure range. More specifically, several cases are stored in the data storing part such as a normal in case of within the standard highest blood pressure range and the standard lowest blood pressure, a high blood pressure in case of higher than the standard highest blood pressure range and a low blood pressure in case of lower than the standard blood pressure range.

In the data storing part 32, also a standard body type range is predetermined and at the same time health conditions are stored as a normal type, an overweight type, an obesity type and a low weight type. In addition, standard ranges of various health data and health conditions are stored.

Since the directly measured data may be displayed in numbers and each health condition corresponding with each figure may be displayed in several ways such as letters or characters, there is an effect that the user can be warned and also another effect that a user eating habit can be improved.

Furthermore, since the measured data accumulatively stored according to each user are displayed in the data storing part 32 in graphs, each user may preserve a data change and take care of their health persistently.

On the other hand, the refrigerator according to the third embodiment also includes a mode switching part 310, and the mode switching part 310 switches from a temperature management mode to a health care mode.

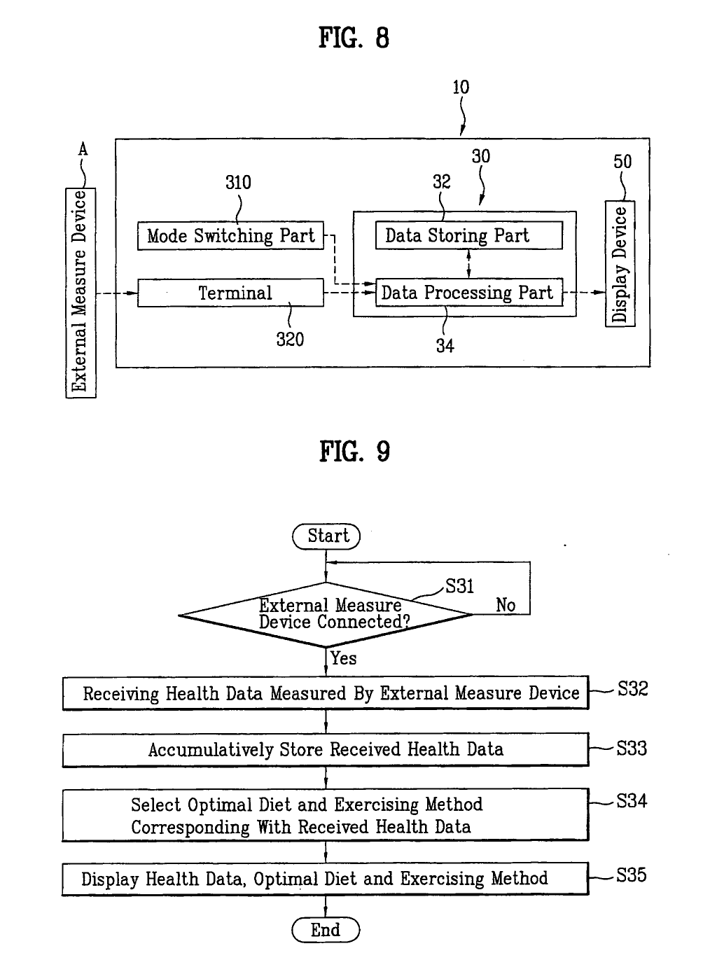

Referring to FIG. 9, a method for controlling the refrigerator according to the third embodiment will be described.

First, it is judged whether the external measure device (A) is connected (S31), and once the external measure device (A) is connected, health data are received (S32).

More specifically, the external measure device (A) is a medical device which can measure a BMI and a blood pressure, and is connected to the terminal 320 formed on the refrigerator door 11. It is determined by the controller 30 whether the external measure device is connected to the terminal 320.

Once it determines that the external measure device (A) is connected to the terminal 320, the controller 30 receives various health data from the external measure device (A) and compares the transmitted health data with the stored standard data of the data storing part 32 to determine a user health condition.

For example, in case that the body type is measured, a health condition is determined as one of a normal, an overweight, an obesity and a low weight by comparing the measured body type and the standard, or in case that a blood pressure is measured, a health condition is determined as one of a normal, a high blood pressure and a low blood pressure by comparing the measured blood pressure with the standard blood pressure.

Hence, the received health data may be accumulatively stored (S33). Then, not only the received health data are stored in the data storing part 32, but also the user health condition compared and determined in the data processing part 34 is stored in the data storing part 32, such that the measured respective user health data and the health condition are persistently managed as well as stored.

An optimal diet corresponding to the received health data is selected (S34). The health data, information relating to the user health condition, the optimal diet and exercises is displayed.

At that time, the display device 50 may display the health data measured for a predetermined time and the health conditions in several ways such as figures, letters, characters and graphs.

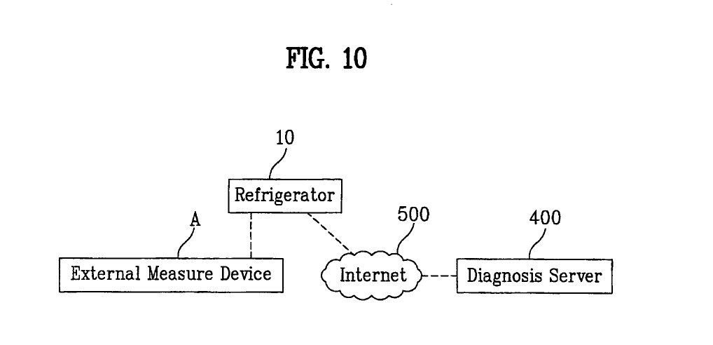

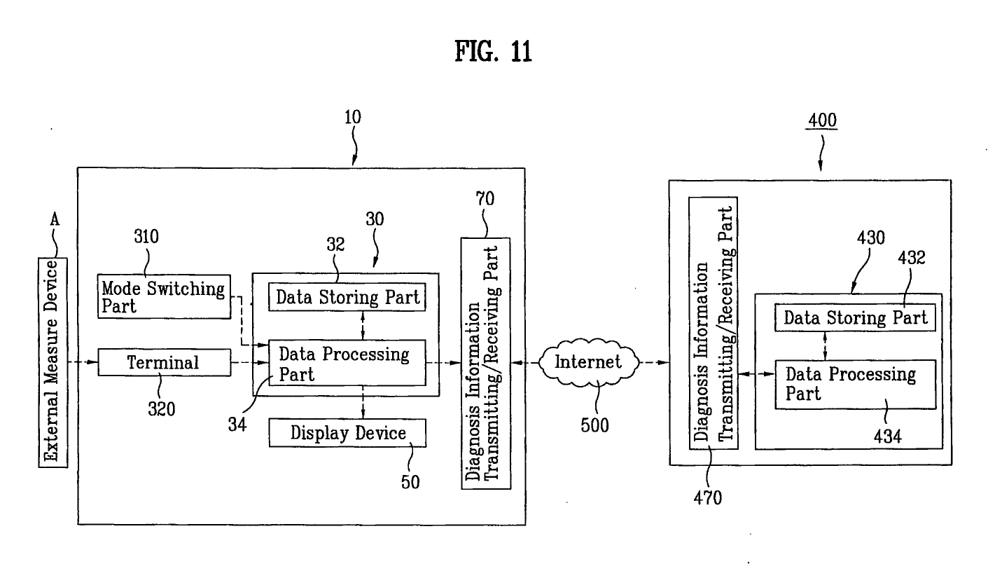

Referring to FIGS. 10 and 11, a refrigerator according to a fourth embodiment of the present invention will be described.

As shown in FIG. 10, a remote control check-up system includes an external measure device (A), a refrigerator 10 and a diagnosis server 400.

The external measure device (A) measures user health data as described in the third embodiment.

The refrigerator receives health data from the external measure device (A), and determines a user health condition based on the health data. Hence, health information including the determined health condition is transmitted to the diagnosis server 400 through the internet 500, and health check-up information received from the health diagnosis server 400 is supplied to a user.

The health diagnosis server 400 receives health information through the internet 500, and performs and/or fills diagnosing and/or prescription. Hence, the health diagnosis server 400 transmits the health diagnosing information to the refrigerator.

Referring to FIG. 11, the remote health check-up system of FIG. 10 will be described in detail.

As described in the third embodiment, the refrigerator according to the fourth embodiment includes a terminal 320 having an external measure device (A) connected thereto, a controller 30, a display device 50 and a mode switching part 310.

The controller 30 includes a data processing part 34 connected to both the mode switching part 310 and the terminal 320 for processing data, and a data storing part 32 connected to the data processing part 34 for storing standard data and measured health data.

The display device 50 displays user health data transmitted by the terminal 320.

The refrigerator further includes a diagnosis information transmitting/receiving part 70 for transmitting/receiving information with the diagnosis server 400.

The diagnosis information transmitting/receiving part 70 transmits the health data received by the terminal to the diagnosis server 400 through a network, and receives the health diagnosis information performing a diagnosis and/or filling a prescription for a user based on the heath data through the diagnosis server 400.

The diagnosis information transmitting/receiving part 70 is a communication port connected to a network such as the internet 500, and is connected through the internet to transmit/receive signals with the diagnosis server 400. The diagnosis information transmitting/receiving may be a modem connected to the internet, and may be an en embedded modem of the refrigerator or an external modem.

The diagnosis information transmitting/receiving part 70 may be a power-line modem. Thus, there is a home server and may be means connected to the home server.

The diagnosis server 400 includes a diagnosis information transmitting/receiving part 470 for communicating with the internet 500 and a diagnosis server controller 430. The diagnosis server controller 430 determines the received health information of the diagnosis information transmitting/receiving part 470 based on the pre-stored standard information. Hence, it performs/fills a diagnosis and/or a prescription, and transmits the generated health diagnosis information to the refrigerator through the diagnosis information transmitting/receiving part 470.

The diagnosis information transmitting/receiving part 470 is corresponding with the diagnosis information transmitting/receiving part 70 provided on the refrigerator.

Next, the diagnosis server controller 430 includes a data processing part 434 and a data storing part 432. The data processing part 434 compares the health information of the diagnosis information transmitting/receiving part 470 with the stored standard information of the data storing part 32, and performs/fills a diagnosis/prescription based on the result of comparison. Hence, the data processing part 434 generates and transmits the health diagnosis information including the above diagnosis/prescription to the data transmitting/receiving part. The data storing part 432 stores the standard information to be compared by the data processing part 434.

The standard information includes a standard data for a predetermined health condition, a standard data for diagnosing disease corresponding with the health condition and a standard data for filling a prescription for the diagnosed disease. Hence, the data processing part 434 analyzes a health condition and/or a disease and/or a body constitution included in the health information, and then performs/fills a diagnosis/prescription for the user.

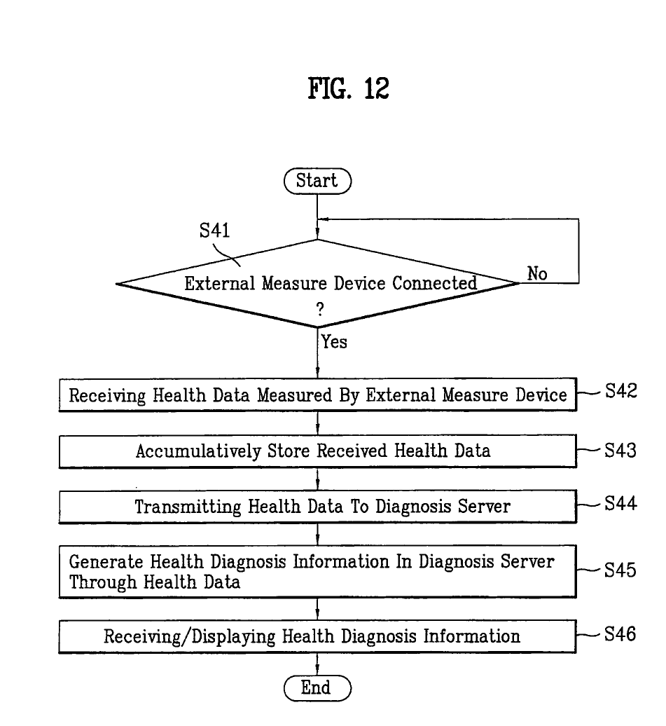

Referring to FIG. 12, a method for controlling the refrigerator according to the fourth embodiment of the present invention will be described.

First, it is determined whether the external measure device (A) is connected (S41), and once the external measure device (A) is connected, health data are received (S42).

More specifically, the external measure device (A) is a medical device which can measure a BMI and a blood pressure, and is connected to the terminal 320 formed on the refrigerator door 11. It is determined by the controller 30 whether the external measure device (A) is connected to the terminal 320.

Once it determines that the external measure device (A) is connected to the terminal 320, the controller 30 receives various health data from the external measure device (A) and compares the transmitted health data with the stored standard data of the data storing part 32 to determine a user health condition.

For example, in case that the BMI is measured, a health condition is determined as one of a normal, an overweight, an obesity and a low weight by comparing the measured BMI and the standard BMI, or in case that a blood pressure is measured, a health condition is determined as one of a normal, a high blood pressure and a low blood pressure by comparing the measured blood pressure with the standard blood pressure.

Hence, the received health data may be accumulatively stored (S43). Then, not only the received health data are stored in the data storing part 32, but also the user health condition compared and determined in the data processing part 34 is stored in the data storing part 32, such that the measured respective user health data and the health condition are persistently managed as well as stored.

Next, the health relating data are transmitted to the diagnosis server 400 (S44). The health data may include user historic diseases pre-stored in the data storing part 32 or user present diseases and/or user body constitution information.

Hence, the health diagnosis information is generated through the health data in the diagnosis server 400. the health diagnosis information is compared and determined with the pre-stored health information, such that the diagnosis/prescription is performed/filled based on the comparison.

Hence, each diagnosis information transmitting/receiving part 70 of the refrigerator 10 and the diagnosis server 400 receives the health diagnosis information, and then the display device 50 displays the health diagnosis information (S46).

Preferably, the display device 50 displays the health diagnosis information in various ways such as figures, letters, characters and graphs to inform the user thereof.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

The refrigerator with the above configuration according to the present invention has following advantageous effects.

First, the refrigerator according to the present invention has an effect that the user is guided for his/her weight and can take care of his/her health more efficiently, because health information is provided to the user by the user health data.

Especially, the user blood sugar, blood pressure and BMI are measured, and the measured health data is displayed, such that the user can take care of his/her health efficiently.

Second, also the refrigerator according to the present invention has another effect that the user can take care of his/her health more efficiently, because the optima diet corresponding with the measured health data is displayed.

Third, the refrigerator according to the present invention has still another effect that the user eating habit may be improved to prevent as well as take care of various diseases, because the terminal capable of connecting various external measure devices thereto is provided and the health information measured by the external measure device is displayed to the user. Thereby, the user health condition may be persistently informed.

Especially, the health data measured by the external measure device is transmitted to the diagnosis server. Hence, the diagnosis and/or prescription information is received, such that the medical advice for the user's present health condition may be provided to prevent as well as take care of diseases.

What is claimed is:

1. A refrigerator for health care comprising:
a display device for displaying user health data; and
a controller for receiving user health data from outside and supplying the user health data to the display device.

2. The refrigerator for health care of claim 1, wherein the health data includes data relating to at least one of a user's weight, body type, face image, body constitution and blood pressure.

3. The refrigerator for health care of claim 1, wherein the controller comprises a data processing part for changing the health data into health information useful for health care.

4. The refrigerator for health care of claim 3, wherein the health information changed by the data processing part includes information relating to at least one of
a user health condition gained by comparison between the health data and predetermined standard data,
a diet corresponding with the health data, and
an exercising method corresponding with the health data.

5. The refrigerator for health care of claim 1, further comprising a measure device for measuring user health data and supplying measured health data to the controller.

6. The refrigerator for health care of claim 5, wherein the measure device comprises a photographing part for receiving a user face image.

7. The refrigerator for health care of claim 5, wherein the measure device comprises a blood sugar sensor for measuring a user blood sugar level.

8. The refrigerator for health care of claim 5, wherein the blood sugar sensor is a not-invasive blood sugar sensor.

9. The refrigerator for health care of claim 7, wherein the blood sugar sensor is provided on the refrigerator door.

10. The refrigerator for health care of claim 1, further comprising a terminal for connecting an external measure device thereto.

11. The refrigerator for health care of claim 10, wherein the controller determines whether the external measure device is connected to the terminal, and receives the health data form the external measure device, once the external measure device is connected to the terminal.

12. The refrigerator for health care of claim 10, wherein the terminal includes a USB port (Universal Serial Bus).

13. The refrigerator for health care of claim 1, further comprising a diagnosis information transmitting/receiving part for transmitting the health data to the diagnosis server through the network and for receiving the health diagnosis information performing/filling a diagnosis/prescription for the user through the diagnosis server based on the health data.

14. The refrigerator for health care of claim 13, wherein the health information displayed by the display device includes health diagnosis information received from the diagnosis information transmitting/receiving part.

15. The refrigerator for health care of claim 1, further comprising a data inputting part for receiving body data inputted by the user.

16. The refrigerator for health care of claim 1, wherein the controller includes a data storing part for accumulatively storing the health data.

17. The refrigerator for health care of claim 16, wherein the health data stored in the data storing part is classified and stored based on respective user information.
